(19) United States
(12) Patent Application Publication
(10) Pub. No.: US 2021/0260248 A1
(43) Pub. Date: Aug. 26, 2021

(54) MEDICAL MEMBRANE MATERIAL

(71) Applicant: National University Corporation Hokkaido University, Sapporo-shi (JP)

(72) Inventor: Naoto Okubo, Sapporo-shi (JP)

(73) Assignee: National University Corporation Hokkaido University, Sapporo-shi (JP)

(21) Appl. No.: 17/261,710

(22) PCT Filed: Jul. 22, 2019
(86) PCT No.: PCT/JP2019/028693

§ 371 Date: Jan. 20, 2021

(30) Foreign Application Priority Data
Jul. 20, 2018 (JP) 2018-137107

(51) Int. Cl. A61L 27/38; A61L 27/54; A61L 15/44

(52) U.S. Cl. CPC A61L 27/3839; A61L 15/44; A61L 27/54

(57) ABSTRACT

The present invention provides a medical film material which comprises a flat-plate-like or film-like demineralized dentin matrix (DDM) that is derived from a removed bovine tooth and completely demineralized, and which has an area falling within the range front 2 to 50 cm². The present invention also provides a method for surgerizing a non-human animal using the medical film material. The present invention also provides a method for producing the medical film material, which comprises thinning and demineralizing a removed bovine tooth to produce a demineralized dentin matrix (DDM) film that is completely demineralized, wherein either one of the thinning procedure and the demineralization procedure may be carried out first.

### FIELD

The present invention relates to a medical membrane material, a method for performing an operation on a non-human animal using the medical membrane material, and a method for producing the medical membrane material.

### BACKGROUND

Teeth are composed of enamel (the surface layer of teeth), dentin, dental pulp, cementum, and periodontal ligament, and mostly composed of enamel and dentin (Patent Literature 1). Dentin includes a natural collagen crosslinked material of high purity produced in a living body, and is therefore considered to be highly useful as a biomaterial. In particular, a demineralized dentin matrix (DDM) obtained by demineralizing a tooth contains collagen as a principal component, and has been attempted to be utilized as a scaffold material for bone formation. For example, Non-Patent Literature 1 shows that, when osteoblasts were proliferated on a DDM (10×5×2 mm), a large number of the osteoblasts adhered to a surface of the DDM and extended.

DDM granules and DDM powder as pulverized products of a DDM are used as transplant materials in dental treatment. Non-Patent Literature 2 shows that a granular DDM was used as a filler for regeneration treatment of an alveolar bone. Non-Patent Literature 2 also describes a block-shaped DDM.

### CITATION LIST

### Patent Literature

Patent Literature 1: Japanese Patent Application Laid-open Publication No. 2007-222811

### Non-Patent Literature

Non-Patent Literature 1: Koga T, et al. (2016) PLoS ONE 11(1): e0147235. doi:10.1371/journal.pone.0147235
Non-Patent Literature 2: In-Woong Um, et al., J Indian Prosthodont Soc 2017; 17:120-7.

### SUMMARY

### Technical Problem

As described above, pulverized products, such as DDM granules and DDM powder, and a DDM block are known as medical materials in which the characteristics of dentin are taken advantage of, in particular, as biomaterials. However, the usage of the DDM pulverized product and the DDM block are limited to a filler, and therefore, a DDM biomaterial having a different shape from those of the pulverized product and the DDM block has been desired to be developed.

As conventional medical materials, various membrane collagen products have been developed. These products are made of collagen derived from bovine or swine dermis, whereas there is no product made of dentin. Furthermore, most of the conventional collagen products include atelocollagen from which telopeptide is removed to decrease antigenicity. This involves the problem that, when such a conventional collagen product is made into a membrane product, a material having sufficient mechanical characteristics for practical use cannot be provided.

To address this, an object of the present invention is to provide a medical membrane material made of a DDM and having sufficient mechanical characteristics for practical use.

### Solution to Problem

To solve the above-described problem, the inventors earnestly studied, and, as a result, found a medical membrane material made of a DDM and having desired mechanical characteristics, and then completed the present invention. According to the present invention, the followings are provided.

[1] A medical membrane material being a demineralized dentin matrix (DDM) and having an area in a range of 2 cm² to 50 cm², the demineralized dentin matrix being derived from extracted bovine teeth, having a plate or membrane shape, and being completely demineralized.

[2] The medical membrane material according to [1], wherein the medical membrane material is used as a transplant material.

[3] The medical membrane material according to [2], wherein the medical membrane material is used to protect, reinforce, or bond an affected site of a non-human animal by bringing the medical membrane material into intimate contact with the affected site.

[4] The medical membrane material according to [1], wherein the medical membrane material is used as a base material for a cell sheet.

[5] The medical membrane material according to any one of [1] to [4], wherein the medical membrane material has a continuous surface.

[6] The medical membrane material according to any one of [1] to [5], wherein a drug is applied to the medical membrane material or the medical membrane material is impregnated with the drug.

[7] A method for performing an operation on a non-human animal, the method including: covering a wound site or an injury site of non-human animal tissue with the medical membrane material according to any one of [1] to [6].

[8] A method for performing an operation on a non-human animal, the method including: filling a wound site or an injury site of non-human animal tissue with a filler, a drug, or a mixture thereof; and covering at least a part of the filler, the drug, or the mixture thereof filled in the wound site or the injury site of the non-human animal tissue, with the medical membrane material according to any one of [1] to [6].

[9] A method for performing an operation on a non-human animal, the method including: connecting a wound site or an injury site of non-human animal tissue via the medical membrane material according to any one of [1] to [6].

[10] A method for producing the medical membrane material according to any one of [1] to [6], the method including: slicing and demineralizing an extracted bovine tooth to obtain a demineralized dentin matrix (DDM) membrane having been completely demineralized, wherein the slicing may be performed prior to the demineralization or the demineralization may be performed prior to the slicing.

[11] The method according to [10], wherein the demineralization is performed by immersing the extracted tooth in a demineralizing solution that is an aqueous solution of any of an inorganic acid, an organic acid, and EDTA.

### Advantageous Effects of Invention

According to the present invention, a medical membrane material made of a DDM and having sufficient mechanical characteristics for practical use can be provided. Because of its flexibility, the completely demineralized DDM can be applied so as to be stuck onto an affected site having a complicated shape.

### BRIEF DESCRIPTION OF DRAWINGS

FIG. 1 is a photograph of molars of cattle (Holstein, adult, female). The photograph illustrates a comparison in size between bovine molars (two molars on the right side: the second molar (M2) and the third molar (M3) are lined up from the left) and human molars (two molars on the left side: the first molar and the second molar are lined up from the left) corresponding to the bovine molars.

FIG. 2A is a diagram to explain a relation between a cutting position (direction) of a tooth and dentinal tubules related to membrane porosity, at a slicing step.

FIG. 2B is a diagram to explain an anatomical difference between a bone and a tooth.

FIG. 3 is a diagram illustrating freeze-drying treatment and reconstitution of a DDM membrane. A photograph on the left illustrates a freeze-dried DDM membrane (9 mm×9 mm). A photograph at the center illustrates a state in which the freeze-dried DDM membrane is reconstituted using phosphate buffered saline (PBS)(−). A photograph on the right illustrates the reconstituted DDM membrane (10 mm×10 mm).

FIG. 4A is a diagram illustrating a comparison between demineralization with a neutral demineralizing solution and demineralization with a weak acid demineralizing solution. In an upper-left photograph, two teeth on the left are bovine anterior teeth; two teeth next to the bovine anterior teeth are bovine mandibular molars; and one tooth on the right is a human molar for comparison. In an upper-right photograph, two teeth on the left are bovine anterior teeth, and two teeth on the right are bovine mandibular molars. Lower photographs with the wording “1 Week” are soft-X-ray photographs. In the lower-left photograph, two teeth on the upper-left side and the lower-left side are bovine mandibular molars; one tooth on the upper-right side is a human molar; and two teeth on the lower-right side are bovine anterior teeth. In the lower-right photograph, two teeth on the upper-left side and the lower-left side are bovine mandibular molars, and two teeth on the upper-right side and the lower-right side are bovine anterior teeth.

FIG. 4B is a diagram illustrating a comparison between demineralization with a neutral demineralizing solution and demineralization with a weak acid demineralizing solution. Photographs with the wording “6 Week” or “7 Week” are soft-X-ray photographs. In the two photographs on the upper left side and the lower-left side, two teeth on the upper-left side and the lower-left side are bovine mandibular molars; two teeth on the upper-right side are bovine anterior teeth; and one tooth on the lower-right side is a human molar. In the upper-right photograph, two teeth on the upper-left side and the lower-left side are bovine mandibular molars, and two teeth on the upper-right side and the lower-right side are bovine anterior teeth. In the lower-right photograph, two teeth are bovine mandibular molars. In FIG. 4B, locations at which completely demineralized teeth are present are illustrated inside frames.

FIG. 4C is a diagram illustrating a comparison between demineralization with a neutral demineralizing solution and demineralization with a weak acid demineralizing solution. Photographs with the wording “12 Week” or “13 Week” are soft-X-ray photographs. In the upper-left photograph, two teeth on the upper-left side and the lower-left side are bovine mandibular molars, and two teeth on the upper-right side and the lower-right side are bovine anterior teeth. In the lower-left photograph, two teeth are bovine mandibular molars. In the two photographs on the upper right side and the lower-right side, teeth are bovine mandibular molars. In FIG. 4C, locations at which completely demineralized teeth are present are illustrated inside frames.

FIG. 5 is a diagram of experimental DDM products of bones (four pieces on the left of a line: upper three pieces are derived from a cancellous bone and lower one piece is derived from a cortical bone) and teeth (three pieces on the right of the line).

FIG. 6 is a diagram illustrating a test of periodontal ligament stem cell proliferation on a DDM membrane. 1. DDM membrane (untreated with CellMatrix Type I Collagen). 2. DDM membrane coated with CellMatrix Type I Collagen.

FIG. 7 is a diagram illustrating a test of periodontal ligament stem cell proliferation on a DDM membrane impregnated with a fibroblast growth factor FGF2. 1. DDM membrane (untreated with FGF2). 2. DDM membrane impregnated with FGF2 having a concentration of 50 ng/mL. 3. DDM membrane impregnated with FGF2 having a concentration of 200 ng/mL.

FIG. 8A is a diagram illustrating a DDM membrane transplant operation on a dog. The upper figure is a photograph illustrating a bone defect site before the operation, and the lower figure is a photograph taken immediately before the transplant of DDM membrane.

FIG. 8B is a diagram illustrating the DDM membrane transplant operation on the dog. The upper figure is a photograph taken immediately after the transplant of the DDM membrane, and the lower figure is a photograph illustrating a comparison between the bone defect site before the operation and the bone defect site on the 21st day after the operation.

FIG. 9 is a diagram illustrating a DDM membrane transplant operation on miniature swine. The upper figure is a photograph taken immediately after the transplant of the DDM membrane; the center figure is a photograph illustrating a state on the third day after the operation; and the lower figure is a photograph illustrating a state on the ninth day after the operation.

FIG. 10 is a diagram illustrating a DDM membrane transplant operation on a dog. a and b are photographs taken before the operation; c is a photograph taken immediately after the transplant of a DDM membrane; and d is a photograph taken 1.5 months after the operation. a and d are X-ray photographs.

FIG. 11 is a diagram illustrating a DDM membrane transplant operation on swine. a is a photograph of a large intestine end before firing, and b is a photograph of a large intestinal anastomosis site taken one week after the operation.

FIG. 12A is a diagram illustrating a DDM membrane transplant operation on swine. The figure illustrates a procedure of side-to-side anastomosis of the small intestine.

FIG. 12B is a diagram illustrating a DDM membrane transplant operation on swine. The upper photograph and the lower photograph are photographs taken one week after the operation and illustrate small intestine anastomosis sites with and without a DDM membrane, respectively.

### DESCRIPTION OF EMBODIMENTS

The following descriptions on the present invention are sometimes given based on typical embodiments and specific examples, but the present invention is not limited to the embodiments and the examples. Note that, in the present specification, a numerical range expressed using “to” indicates a range including a numerical value preceding “to” as the lower limit and including a numerical value following “to” as the upper limit.

[Medical Membrane Material]

One embodiment of the present invention relates to a medical membrane material being a demineralized dentin matrix (DDM) and having an area in a range of 2 cm² to 50 cm², the DDM being derived from extracted bovine teeth, having a plate or membrane shape, and being completely demineralized. Hereinafter, the medical membrane material according to the present invention is sometimes referred to as a DDM membrane. The DDM membrane according to the present invention is based on the inventors' findings that a membrane made of a DDM produced by cutting an extracted bovine tooth in a plate or membrane shape and completely demineralizing the extracted bovine tooth has advantageous characteristics as a medical material.

The medical membrane material according to the present invention is a plate- or membrane-shaped DDM and produced by cutting dentin in a plate or membrane shape. The plate or membrane shape means a straight flat plate or membrane shape, and examples thereof include a plate or membrane shape curved or warped to the extent that such shape neither interferes with sticking the medical membrane material on an affected site (for example, a wound site or an injury site) and nor interferes with cell culture.

Regarding the plate or membrane shape, only in terms of the thickness of the DDM, a shape having a comparatively large thickness is just expressed as a plate shape, whereas a shape having a comparatively small thickness is just expressed as a membrane shape, and there is no essential difference between the plate shape and the membrane shape. The DDM (demineralized dentin matrix) is obtained by completely demineralizing the dentin of an extracted bovine tooth. A component of the dentin will be described later.

The medical membrane material according to the present invention preferably has a thickness in a range of 10 μm to 2000 μm. The thickness of the medical membrane material according to the present invention can be 10 μm or more, 50 μm or more, 100 μm or more, and 200 μm or more, and furthermore can be 2000 μm or less, 1900 μm or less, 1800 μm m or less, 1700 μm or less, 1600 μm or less, 1500 μm or less, 1400 μm or less, 1300 μm or less, 1200 μm or less, 1100 μm or less, 1000 μm or less, 900 μm or less, 800 μm or less, 700 μm or less, 600 μm or less, 500 μm or less, 400 μm or less, and 300 μm or less. In accordance with the uses of the medical membrane material, the thickness can be suitably adjusted. For example, in the case where the medical membrane material is desired to be absorbed into a living body in a short time, the thickness can be reduced. In contrast, as in the case of being used as a protective material, when the medical membrane material is desired to be maintained for a long period of time, the thickness can be increased. Note that, as described in detail later, when the medical membrane material according to the present invention is used as a transplant material, the thickness is preferably in a range of 100 μm to 2000 μm, whereas, when the medical membrane material according to the present invention is used as a base material for a cell sheet, the thickness is preferably in a range of 10 μm to 300 μm.

The medical membrane material according to the present invention has elasticity and/or toughness. This is because the medical membrane material hardly contains a mineral component. The elasticity indicates a property such that, when an object transformed by external force is released from the external force, the object tends to return to its original shape. The medical membrane material according to the present invention becomes distorted when a stress is applied thereto, but, can return to its original shape when released from the stress. For example, the medical membrane material has a property such that, when the top face of the membrane is pressed with a fingertip, the top face of the membrane is dented, and, when released from the pressurization by the fingertip, the top face of the membrane returns to its original flat shape that is a shape before the pressurization. The toughness is a property such that a material is tough, specifically, is resistant to external force and hard to be broken. The medical membrane material according to the present invention has a property such that, when the top face of the membrane is strongly pressed with a fingertip, the top face of the membrane is dented, but, the membrane is hard to be broken. In the present specification, “having flexibility” indicates having elasticity and/or toughness.

The medical membrane material according to the present invention is made of dentin of an extracted bovine tooth. The dentin is a hard tissue constituting most regions of a tooth, and is present to support dental pulp inside the tooth and enamel and cementum surrounding the tooth. The dentin is formed by calcification of an organic matrix synthesized and secreted from odontoblast. In the composition of the dentin, a calcified mineral component amounts to 70% of the entirety, the calcified mineral component being mostly composed of hydroxyapatite (a crystal made of phosphoric acid and calcium), and furthermore, moisture amounts to 10%, and an organic component amounts to 20%. Since the mineral component of the dentin is dissolved by demineralization, a component remaining after the demineralization is the organic component. In the organic component, collagen amounts to approximately 90% and noncollagenous protein amounts to the remaining approximately 10%. It is known that the noncollagenous protein includes dentin sialophosphoprotein at the largest ratio, and after synthesis in an odontoblast, dentin sialoprotein, dentin glycoprotein, and dentin phosphoprotein are produced from the dentin sialophosphoprotein.

An extracted bovine tooth can be a tooth extracted after slaughter or by treatment. Examples of a tooth type that can be used as a raw material for the DDM membrane include deciduous teeth and permanent teeth (an incisor, a cuspid, a premolar and a molar). The DDM membrane can be more efficiently produced from a larger tooth than a smaller tooth, and therefore, a molar is preferably used as a raw material for the DDM membrane, and a premolar or molar is particularly preferably used. The most preferable bovine tooth is a healthy extracted bovine premolar or molar.

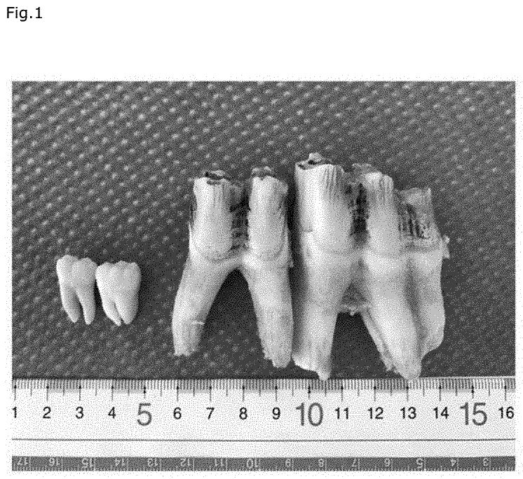

The medical membrane material according to the present invention is derived from a tooth of a large-sized mammal (such as cattle, a horse, swine, sheep, or a goat) having teeth containing dentin in large amount, particularly derived from a bovine tooth. Many cattle are bred as beef cattle or dairy cattle, and teeth thereof are discarded without being used, and therefore are stably available in large amount at low cost. Furthermore, among large-sized mammals, cattle have particularly large teeth, and therefore, a large amount of dentin can be obtained from one tooth, and a DDM membrane having a large surface area can be obtained. FIG. 1 is a diagram illustrating a comparison in size between bovine molars and human molars.

The medical membrane material according to the present invention can be used in autologous treatment, allogeneic treatment, and xenogeneic treatment. The use of the medical membrane material according to the present invention in autologous treatment means that the medical membrane material which is a DDM membrane produced from an extracted bovine tooth is used for treatment for the cattle. Examples of the use of the medical membrane material according to the present invention in allogeneic treatment may include that bovine teeth are collected (for example, teeth bank) to produce a medical membrane material and used for treatment of cattle other than a donor. The use of the medical membrane material according to the present invention in xenogeneic treatment means that a medical membrane material is produced from an extracted bovine tooth and used for treatment of animals, other than cattle, or of humans.

The medical membrane material according to the present invention is completely demineralized dentin. The completely demineralized dentin indicates dentin containing no mineral component or dentin demineralized to the extent that the dentin contains little mineral component. Whether dentin contains no mineral component or contains little mineral component can be confirmed, during production, by using a soft-X-ray photography device manufactured by Softex by the fact that an X-ray impermeable portion has disappeared completely or nearly completely. Note that partially demineralized dentin indicates dentin in which a mineral component of the dentin partially remains and the composition of which is approximately 5% to 70% of mineral component, approximately 20% to 95% of collagen, and approximately 5% to 10% of water.

The completely demineralized dentin contains little mineral component, but contains collagen as a principal component, and is excellent in flexibility. The collagen contained in the medical membrane material according to the present invention is Type I collagen. This is because the matrix of the dentin serving as a raw material is Type I collagen.

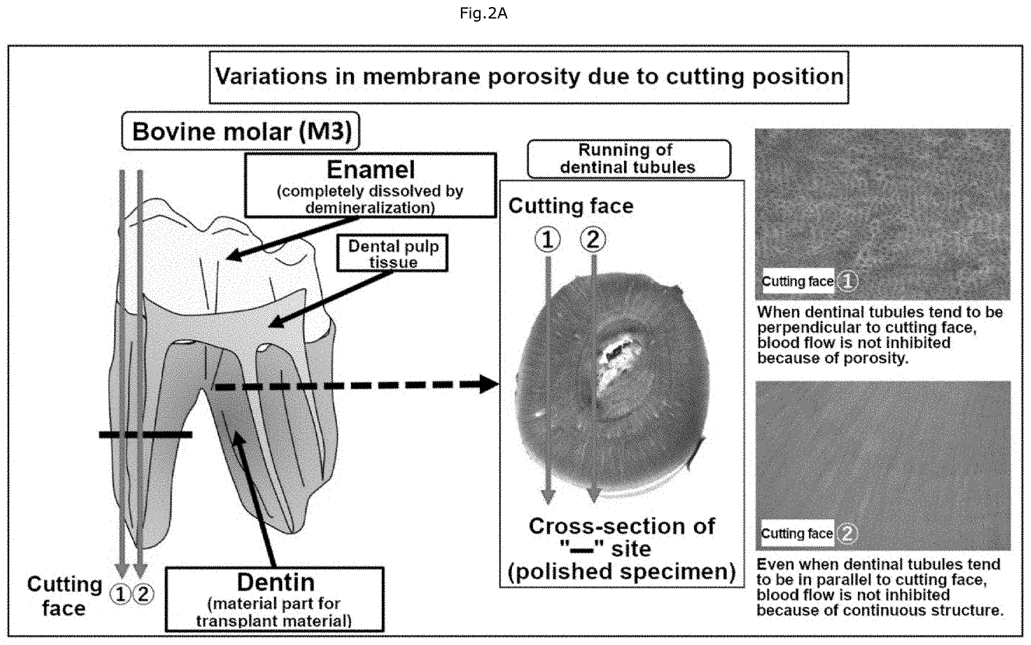

The medical membrane material according to the present invention is porous. In both human teeth and teeth of mammals other than humans, dentinal tubules run densely (regularly) from the outer surface of dentin toward the center (pulp cavity) (FIG. 2B). With this structure, even when a tooth is processed into any shape (such as a membrane shape or a granular shape), the dentin always become a dense transplant material having continuous pores (photographs in FIG. 2A). The medical membrane material according to the present invention can have 5000 to 15000 continuous-pores/mm² on the top face of the membrane, the continuous-pores having a pore size in a range of 0.8 μm to 15 μm and being continuous to the bottom surface of the membrane.

The medical membrane material according to the present invention can have a membrane area in a range of 0.25 cm² to 50 cm². The membrane area is preferably in a range of 2 cm² to 50 cm². The membrane area can be suitably adjusted in accordance with the uses of the DDM membrane. The membrane area is defined as an area of a face along the plane direction of the membrane when the medical membrane material is viewed as the entirety of the membrane. The maximum membrane area depends on the size of an extracted tooth serving as a material. For example, when a bovine premolar or molar is used as a raw material, a DDM membrane having a membrane area of 50 cm² at the maximum can be produced. The medical membrane material according to the present invention can have a membrane area of 0.25 cm² or larger, 0.5 cm² or larger, 1 cm² or larger, 1.5 cm², 2 cm² or larger, 3 cm² or larger, 4 cm² or larger, and 5 cm² or larger, and furthermore 50 cm² or smaller, 40 cm² or smaller, 30 cm² or smaller, 25 cm² or smaller, 20 cm² or smaller, 15 cm² or smaller, 10 cm² or smaller, and 5 cm² or smaller. In accordance with the uses of the medical membrane material, the membrane area can be suitably adjusted.

The medical membrane material according to the present invention can have a continuous surface form. The continuous surface form is a form not having any joint and any missing part (that is, a hole) that impair mechanical characteristics required as the medical membrane material or hinder the membrane from sticking on or covering an affected site. The hole mentioned herein typically indicates a large missing part of the membrane originated from a pulp cavity, for example, and does not indicate a micropore like a continuous pore formed by dentinal tubules. The medical membrane material according to the present invention that has a continuous surface form is preferably used to protect, reinforce, or bond an affected site by bringing the medical membrane material into intimate contact with the affected site.

The medical membrane material according to the present invention allows a drug to be applied thereto, or can be impregnated with the drug. The drug that is to be applied to the DDM membrane or with which the DDM membrane is impregnated can be selected in accordance with the uses of the DDM membrane, and examples of the drug include an epidermal growth factor, a fibroblast growth factor, an insulin-like growth factor, a hepatocyte growth factor, a bone morphogenetic factor, laminin, fibrin, elastin, and fibronectin.

When used as a medical material, especially as a biomaterial, the DDM membrane has advantageous characteristics: 1) the DDM membrane has sufficient mechanical characteristics for practical use, particularly elasticity and/or toughness, whereas the DDM membrane contains Type I collagen as a principal component and is therefore easily enzymatically degraded in a body after transplant; 2) a time period over which the DDM membrane is subjected to biolysis or absorbed can be adjusted by controlling the thickness of the DDM membrane; 3) the DDM membrane has porosity owing to a dentinal tubule structure, and therefore has high body fluid permeability, and can complement a defect in conventional membrane materials, namely, blood flow inhibition; 4) the DDM membrane contains Type I collagen as a principal component, and therefore migration of cells can be expected at an early stage and the DDM membrane has the effect of promoting healing; and 5) the DDM membrane adsorbs a drug, such as a cell growth factor, and thereby the sustained-release effect can be expected. Therefore, the DDM membrane can be used as a medical membrane material, such as a medical membrane transplant material, a cell proliferation scaffold, and a base material for a cell sheet.

[Transplant Material]

The medical membrane material according to the present invention can be used as a transplant material. The transplant material is a biomaterial that can be used for surgical treatment. The medical membrane material according to the present invention is capable of protecting, reinforcing, or bonding an affected site by transplanting the medical membrane material into the affected site, specifically by bringing the membrane material into intimate contact with the affected site. In the present specification, an affected site means a site of a human or a non-human animal that is desired to be subjected to treatment, and includes a wound site and an injury site. Bringing the medical membrane material into intimate contact with an affected site means that the medical membrane material is arranged at a location very close to the affected site so as to stick to the affected site, and does not mean that any gap including a minute gap is not present between the medical membrane material and the affected site.

For example, by using the medical membrane material according to the present invention as a transplant material, an affected site can be protected by transplanting the medical membrane material into a soft tissue of a treatment target site. In addition, the medical membrane material according to the present invention can be used for the purpose of direct transplanting into a hard tissue of a treatment target site and also for the purpose of, when a hard-tissue defect site is filled with another granular transplant material, packaging to stably immobilize the granular transplant material at an affected site, in other words, for the purpose of covering the affected site after the filling. Furthermore, when the medical membrane material according to the present invention is used as a transplant material, there is a possibility that the medical membrane material can inductively promote tissue regeneration in a transplant site. Furthermore, the medical membrane material according to the present invention can be transplanted into a treatment target, together with cells proliferated on the medical membrane material, and therefore can be used as a cell proliferation scaffold for transplant. The cell proliferation scaffold is an artificial extracellular matrix that is necessary until cells become able to create their own extracellular matrix at a tissue defect site.

In the case where an affected site is present in a surface of a living body tissue, a transplant of the medical membrane material according to the present invention can be performed in such a manner that the membrane material is brought into intimate contact with the affected site to cover the affected site, and furthermore, the membrane material is fixed as necessary. In the case where an affected site is present inside a living body tissue, a transplant of the medical membrane material according to the present invention can be performed in such a manner that the affected site inside the living body tissue is exposed such as by making an incision in the living body tissue, and the membrane material is brought into intimate contact with the affected site to cover the affected site, and furthermore the membrane material is fixed as necessary, and then, the affected site exposed by the incision or the like is returned to its original state, such as by closing the incision by suturing or the like.

Examples of the application of the medical membrane material according to the present invention as a transplant material for treatment include, but not limited to, application in the periodontal therapy field (for example, application as a patch to an extraction socket), application in the dermatology field (for example, application as a patch or a dressing material to decubitus ulcers, a burn site, or the like), application in the gastroenterological surgery field (for example, application as a preventive material against postoperative leaks via supporting for engrafting a sutured site, such as a sutured site of a perforated gastrointestinal tract portion), application in the cardiovascular surgery field (for example, application for pre-packaging of, for example, a portion of a thinner blood vessel wall, in other words, application for strength reinforcement by covering), and application to a bone fracture site (for example, application for the promotion of healing of a bone fracture by packaging a fracture site, that is, covering a fracture site after the bone fracture is reduced).

When the medical membrane material according to the present invention is used as a transplant material, the medical membrane material preferably has a thickness in a range of 100 μm to 2000 μm. The medical membrane material has sufficient mechanical characteristics as a transplant material, has a characteristic that, when transplanted into a living body, the medical membrane material sufficiently sticks to an affected site by incorporating surrounding moisture (such as blood) into a dentinal tubule structure of the medical membrane material, and has sufficient flexibility as a transplant material. Furthermore, the medical membrane material according to the present invention can package, for example, a granular transplant material because of its sufficient mechanical characteristics for practical use and its sufficient flexibility. Furthermore, the medical membrane material is not broken even when fixed to a transplant bed, for example, by a titanium pin or a suture. Furthermore, even when bridging of a bone defect site is performed using the medical membrane material according to the present invention alone, healing can be completed without dehiscence of the backing epithelium at the bone defect site. Furthermore, although collagen membranes have been used for closing a perforated portion of intestines or the likes, the medical membrane material according to the present invention can be used in place of such conventional collagen membranes. Conventional collagen membranes have low strength and therefore often fail to close a perforation, whereas the medical membrane material according to the present invention has sufficient mechanical characteristics for practical use and also functions as a scaffold for cells, and therefore can be applied for medical use, as a good patch material.

When the medical membrane material according to the present invention is used as a cell proliferation scaffold for transplant, the medical membrane material preferably has a thickness in a range of 100 μm to 300 μm. Such medical membrane material is not easily broken, and, for example, when the medical membrane material is used as a base material for a myocardial cell sheet and transplanted into the heart, the medical membrane material does not inhibit pulsation.

Since the medical membrane material according to the present invention is porous, the supply of a body fluid to a transplant site is not inhibited, and hence it is considered that a bacterial infection of the transplant site hardly occurs. This is because, after transplant into an operation site, only a serous component in blood containing a large amount of an antibacterial substance, such as immunoglobulin, supplied from a transplant bed (mother bed) contact site on the front side of the membrane promptly moves to a side opposite to a transplant membrane by a semipermeable membrane action owing to the diameter (10 μm or smaller) of a dentinal tubule, and the serous component covers the entirety of the membrane, so that antibacterial properties are achieved. With such properties, the medical membrane material according to the present invention can overcome susceptibility to infection, which is a disadvantage of the conventional transplant materials. In the case of a typical membrane material, as the thickness of the membrane material is increased, the risk of blood flow inhibition becomes higher, whereas, even when the medical membrane material according to the present invention has an increased thickness, body fluid permeability is kept high because of the dentinal tubule structure, so that there is no risk of blood flow inhibition. The medical membrane material is capable of adsorbing a nutritional factor inside tubules of the dentinal tubule structure, and therefore not only functions as a scaffold for cells, but also supports cell proliferation, and thereby achieves a regeneration promotion effect.

The medical membrane material according to the present invention is an assemblage of Type I collagen, and is therefore excellent also as a scaffold to which cells adhere. When the medical membrane material according to the present invention is used as a cell proliferation scaffold for transplant, the medical membrane material can be transplanted into a treatment target, together with cells proliferated on the medical membrane material. The medical membrane material is capable of adsorbing a nutritional factor that is thought to be ideal for tissue of a transplant destination. For example, by using the transplant membrane on which osteoblasts proliferate and furthermore into which BMP2 serving as a bone morphogenetic factor is absorbed, a time period of healing of the fracture site can be considerably shortened. Examples of cells that can be cultured using the medical membrane material according to the present invention as a scaffold material include, but not limited to, induced pluripotent stem (iPS) cells, embryonic stem (ES) cells, and other tissue stem cells (such as mesenchymal stem cells and periodontal ligament stem cells).

As another embodiment, the present invention can provide a method for performing an operation on a non-human animal or a human, the method including covering a wound site or an injury site of tissue of the non-human animal or the human with the above-described medical membrane material. In addition, as another embodiment, the present invention can provide a method for performing an operation on a non-human animal or a human, the method including connecting a wound site or an injury site of tissue of the non-human animal or the human via the above-described medical membrane material.

The wound site is a site of a wound caused by an operation. The injury site is a site of an injury caused by an accident or a disease, for example. The non-human animal is an animal other than humans, in particular an animal raised as a pet or livestock, such as a dog, a cat, a rabbit, a mouse, a horse, cattle, a goat, or sheep. Covering a wound site or an injury site with the medical membrane material is to cover at least a part of the wound site or the injury site with at least one sheet of the medical membrane material, and is preferably to cover the entirety of the wound site or the injury site with the medical membrane material, although depending on the size of the wound site or the injury site. The connection of wound sites or injury sites via the medical membrane material is to interpose via the medical membrane material so as to bring at least one sheet of the medical membrane material into contact with at least a part of a connection face of the wound site or the injury site, and join the wound sites or the injury sites, and the medical membrane material is preferably involved in a large part of, particularly the entirety of the connection face of the wound site or the injury site, although depending on the size of the wound site or the injury site.

The method for performing an operation on a non-human animal or a human may further include fixing the medical membrane material after covering with the membrane material. In the case where the medical membrane material is transplanted into a hard tissue, the membrane material after the transplant incorporates surrounding moisture (such as blood) into the dentinal tubule structure and sticks to a transplant site, and thereby naturally fixed to the transplant site, which eliminates the need for any active fixing approach. In the case where the medical membrane material is transplanted into the interior of a living body tissue, for example, when an incision is made in the living body tissue to expose an affected site thereinside and then the affected site is covered with the membrane material and then the exposed affected site is returned to its original state, the medical membrane material is often eventually fixed by the surrounding living body tissue, which eliminates the need for any active fixing approach. In contrast, in the case where the medical membrane material is transplanted into a flexible soft tissue such as a digestive tract, the medical membrane material is desirably fixed.

The fixation of the medical membrane material to a wound site or an injury site and the connection of the wound site or the injury site via the medical membrane material can be performed by suturing with a suture, fixation using a medical stapler, or bonding using a medical tape.

The method for performing an operation on a non-human animal or a human can be performed in the periodontal therapy field (for example, a bone development operation such as the guided tissue regeneration (GTR) method, a sinus lift operation, and extraction of a tooth, such as a wisdom tooth), in the dermatology field (for the purpose of protecting a raw surface after debridement operation on an infection tissue), in the gastroenterological surgery field (for example, digestive tract anastomosis and perforated digestive tract closure), in the orthopedic surgery field (for example, fracture reduction), and in the cardiovascular surgery field (for example, artificial blood vessel replacement). When the medical membrane material according to the present invention is used as a cell proliferation scaffold for transplant, the medical membrane material can be generally applied to stem cell sheet transplant operations in which, for example, the medical membrane material is transplanted into the heart as a myocardial sheet produced by induction of differentiation of an iPS cell or a tissue stem cell.

As still another embodiment, the present invention can provide a method for performing an operation on a non-human animal or a human, the method including: filling a wound site or an injury site of non-human animal tissue or human tissue with a filler, a drug, or a mixture thereof; and covering at least a part of the filler, the drug, or the mixture thereof filled in the wound site or the injury site of the non-human animal tissue or the human tissue, with the medical membrane material. The wound site is a site of a wound caused by an operation. The injury site is a site of an injury caused by an accident or a disease, for example. The non-human animal is an animal other than humans, in particular an animal raised as a pet or livestock, such as a dog, a cat, a rabbit, a mouse, a horse, cattle, a goat, or sheep.

Filling a wound site or an injury site with a filler, a drug, or a mixture thereof is preferably performed after a cleaning of the wound site or the injury site (debridement). Examples of the filler to be filled in the wound site or the injury site include DDM granules, DDM powder, DDM blocks, freeze-dried bone allograft (FDBA), demineralized freeze-dried bone allograft (DFDBA), hydroxyapatite, calcium hydroxide, and heterologous-bones-derived bone mineral transplant materials (for example, Bio-oss (registered trademark)). Examples of the drug to be filled in the wound site or the injury site include antibiotics (for example, tetracycline ointment) and cell growth factors (FGF2 preparation: Fiblast Spray, for example). Covering the wound site or the injury site with the medical membrane material is to cover at least a part of the wound site or the injury site with at least one sheet of the medical membrane material. Although depending on the size of the wound site or the injury site, the entirety of the wound site or the injury site is preferably covered with the medical membrane material. This is because the filler, the drug, or the mixture thereof that have been filled is prevented from being exposed above the wound site or the injury site.

The method for performing an operation on a non-human animal or a human may further include fixing the medical membrane material after covering with the membrane material. In the case where the medical membrane material is transplanted into a hard tissue, the membrane material after the transplant incorporates surrounding moisture (such as blood) into the dentinal tubule structure and sticks to a transplant site, and thereby naturally fixed to the transplant site, which eliminates the need for any active fixing approach. In the case where the medical membrane material is transplanted into the interior of a living body tissue, for example, when an incision is made in the living body tissue to expose an affected site thereinside and then the affected site filled with a filler is covered with the membrane material and then the exposed affected site is returned to its original state, the medical membrane material is often eventually fixed by the surrounding living body tissue, which eliminates the need for any active fixing approach. In contrast, in the case where the medical membrane material is transplanted into a flexible soft tissue such as a digestive tract, the medical membrane material is desirably fixed.

Fixation of the medical membrane material to a wound site or an injury site can be performed by suturing with a suture, fixation using a medical stapler, or bonding using a medical tape.

In one embodiment, the method for performing the operation on a non-human animal or a human can be performed particularly in the periodontal therapy field. An alveolar bone site in which bone resorption has been caused by a congenital disease (such as gnathopalatoschisis) or a periodontal disease is filled with a filler, a drug, or a mixture thereof and covered with the above-described medical membrane material, and the membrane material is fixed, whereby the membrane material protects the transplanted substances to promote regeneration of the alveolar bone, and at the same time, plays a role of a scaffold for soft tissue to be backed, and thereby has the effect of preventing the dehiscence of a soft tissue suture site. With these functions, a disease, a disorder, or a symptom in the periodontal therapy field can be treated or prevented.

[Base Material for Cell Sheet]

The medical membrane material provided by the present invention can be used as a base material for a cell sheet in cell culture or regenerative therapy. The cell sheet indicates layered cells cultured with high density on a base material or a support, and is used in regenerative therapy that renatures a damaged biological function by using a stem cell or the like. The base material for the cell sheet is sometimes transplanted into a treatment target, together with cells proliferated on the base material. Therefore, the base material for the cell sheet is required to have bioabsorbability, cell adhesiveness, and form-stability. Furthermore, the base material needs to be porous in order to supply sufficient nutrition to tissues or cells. The medical membrane material provided by the present invention has bioabsorbability, cell adhesiveness, and form-stability and is porous, and is therefore advantageous as the base material for the cell sheet.

When the medical membrane material according to the present invention is used as the base material for the cell sheet, the medical membrane material preferably has a thickness in a range of 10 μm to 300 μm. It is thought that such medical membrane material is not easily broken, and, because of the dentinal tubule structure, the medical membrane material is porous, and therefore the medical membrane material does not inhibit body fluid permeation and nutritional exchange even when it is thick, and furthermore a state in which that a body fluid such as a nutritional factor is allowed to be highly permeable from the back side of the membrane is provided. There is no conventional medical membrane material available that has a thickness of 300 μm or less, has a porous structure derived from collagen and having a regular arrangement, and causes neither shrinkage nor a decrease in mechanical strength even when the medical membrane material comes into contact with a body fluid (blood).

When the medical membrane material according to the present invention is used as the base material for the cell sheet, a DDM membrane can be obtained by immersing an extracted tooth in a demineralizing solution that is an aqueous solution of any of an inorganic acid, an organic acid, and EDTA to demineralize the extracted tooth. In one embodiment of the present invention, when the medical membrane material is used as the base material for the cell sheet, a DDM membrane obtained by the demineralization using an EDTA aqueous solution (neutral) is preferably used. This is because, in a cell culture experiment, the DDM membrane obtained by demineralization using the EDTA aqueous solution (neutral) more promptly causes cell adherence than a DDM membrane obtained by demineralization using an inorganic or organic acid aqueous solution.

[Comparison with Conventional Products]

Conventional medical membrane materials can be classified into two types in terms of raw material. One type is a Type I collagen membrane (for example, Koken Tissue Guide) that is produced by preparing atelocollagen and interweaving it again. This type is very weak in strength, and shrinks when coming into contact with blood, and therefore it is thought that this type cannot be used as a transplant material for uses requiring strength (for example, bridging of a parenchyme defect site). Furthermore, this type has the characteristics of being easily degraded and absorbed and being hardly infected even when exposed somewhat. The other type is a membrane (for example, GC membrane) obtained by weaving an artificial raw material. Although being made of the artificial raw material, this type is weak in strength and many of this type have rather a low affinity for blood and are blood repellent. When exposed, this type is easily infected. Both the two types have no action of coming into intimate contact with a wound and is poor in usability.

In contrast, the medical membrane material according to the present invention includes, as a principal component, Type I collagen serving as a scaffold that is most important for cells, and the greatest characteristic of the medical membrane material is that collagen is used as it is without degradation into atelocollagen. Furthermore, the medical membrane material is made of a natural tooth, and therefore has sufficient mechanical characteristics for practical use and sufficient flexibility. Therefore, the medical membrane material can be sewn onto an affected site with a suture. The medical membrane material is porous because of its dentinal tubule structure, and therefore has a very high affinity for blood, does not inhibit blood flow which is important for tissue regeneration, and adheres to an affected site and accordingly has high usability. The medical membrane material can adsorb a nutritional factor, and thereby can promote tissue regeneration. Furthermore, the medical membrane material is highly resistant to infection.

The characteristics of the conventional medical membrane materials and the medical membrane material according to the present invention are listed in the table below.

TABLE 1

| | | Conventional | | |
|---|---|---|---|---|
| | | | Artificial | Present |
| | | Atelocollagen | synthetic | invention |
| | | membrane | membrane | DDM membrane |
| Reaction | Affinity | good | poor | very high |
| to blood | | spongy | (hydrophobic | affinity |
| | | | and repellent | because of |
| | | | to blood) | porosity |
| | | | no pore, wall | coated with |
| | | | | body fluid |
| | Stability | has low | becomes | has physical |
| | | stability and | fragile when | properties that |
| | | remarkably | absorbing blood | do not change |
| | | shrinks | difficult to | even when |
| | | cannot be | be fixed by | exposed to |
| | | fixed by suture | suture | blood |
| | | cannot be | cannot be | tough and can |
| | | fixed with | fixed with | be fixed by |
| | | titanium pin | titanium pin or | suture |
| | | | the like | can be easily |
| | | | | fixed with |
| | | | | titanium pin |
| Adhesion to wound | | no adhesion | no adhesion | good adhesion |
| Usability | | poor | poor | excellent |
| Effect as scaffold | | has no effect | has no effect of | has the effect |
| for cells | | because of | retaining cells | of stabilizing |
| (stability of wound | | shrinkage by | because the | a wound because |
| site) | | blood | membrane is | the membrane |
| | | | not collagen | has strong |
| | | | | physical |
| | | | | properties and |
| | | | | contains |
| | | | | collagen as a |
| | | | | principal |
| | | | | component |
| Nutritional factor | | no effect | no effect | effective: |
| retention effect | | | | contribution to |
| | | | | promotion of |
| | | | | wound |
| | | | | regeneration |
| Infectiousness | | hard to be | infectible | resistant to |
| | | evaluated | easily infected | infection |
| | | because of its | when exposed | resistant to |
| | | solubility | | infection even |
| | | | | exposed |
| Absorptive property | | quick | quick | rather slowly |
| | | causes no | induces | absorbed |
| | | impairment | inflammatory | because made |
| | | | reaction when | of highly |
| | | | absorbed | cross-linkable |
| | | | | collagen |
| | | | | causes little |
| | | | | impairment |

[Production Method]

The present invention can provide a method for producing the above-described medical membrane material, the method including slicing and demineralizing an extracted bovine tooth to obtain a demineralized dentin matrix (DDM) membrane completely demineralized, wherein the slicing may be performed prior to the demineralization or the demineralization may be performed prior to the slicing.

The slicing of the extracted tooth means slicing the tooth thinly to produce thin slices. The thickness of the slice can be suitably adjusted in accordance with uses of the medical membrane material, and can be 10 μm to 2000 μm. The cutting direction of the tooth can be, but is not limited to, a direction in parallel to the major axis of the tooth as illustrated in FIG. 2A, so as to achieve the largest cut surface of the tooth.

Alternatively, depending on uses of the medical membrane material (for example, when the medical membrane material is used to be placed on the bottom surface in a culture dish so as to more easily seed cells), an anterior tooth is cut perpendicular to the major axis thereof and thereby can be processed into a nearly-round-shaped membrane. In the case of producing the medical membrane material having a continuous surface shape, in order to avoid the formation of a hole originated from a pulp cavity, a tooth is preferably cut at a location at which there is no pulp cavity or at a location at the center of which a pulp cavity is not present. Furthermore, before or after the slicing, chamfering work or the work of cutting off an unnecessary portion can be performed so that the DDM membrane has a fixed size and a fixed shape (for example, a quadrangular or round shape).

The slicing of an extracted tooth may be performed before the later-mentioned demineralization or may be performed after the demineralization. For example, based on the characteristics of a slicing machine to be used for slicing the tooth, an order in performing the slicing and the demineralization can be determined. In other words, when the slicing machine is more suitable for cutting a hard object than a soft object, the slicing can be performed before the demineralization. In the case where the slicing of the tooth is performed after the demineralization, a slicing machine, such as a microtome (for example, RETORATOME REM-710: Yamato Kohki Industrial CO., LTD.) or a slicer, can be used for the slicing. In the case where the slicing of the tooth is performed before the demineralization, a slicing machine, such as a diamond cutter (for example, a precision cutter, IsoMet High Speed & High Speed Pro: BUEHLER) or a band saw (a micro-cutting machine, BS-300CP: Meiwafosis Co., Ltd.), can be used for the slicing.

The demineralization of an extracted tooth is a treatment for remove a mineral component of the tooth. The demineralization of an extracted tooth can be performed by various methods, for example, by immersing an extracted tooth in a demineralizing solution. Examples of the demineralizing solution to be used include an inorganic acid and an aqueous solution thereof, an organic acid and an aqueous solution thereof, and an EDTA aqueous solution. Examples of the inorganic acid to be used as the demineralizing solution include, but not limited to, nitric acid and hydrochloric acid. Examples of the organic acid to be used as the demineralizing solution include, but not limited to, formic acid, acetic acid, citric acid, lactic acid, and a mixture thereof. The concentration of the inorganic acid or the organic acid in the demineralizing solution can be suitably determined in terms of an amount required to dissolve an apatite component derived from an extracted tooth, and for example, an aqueous solution having a concentration of 5% to 30% can be used. The liquid temperature of the demineralizing solution can be 4° C. to 60° C., for example. A period of time required for the demineralization differs depending on the concentration, liquid temperature and pH of the demineralizing solution and the size and shape of an extracted tooth, and therefore cannot be generalized, but, in the case where an extracted bovine tooth is demineralized by an inorganic acid or an aqueous solution thereof in a state in which the size or shape of the tooth is as it is, 6 weeks or more are usually required for the demineralization. In contrast, in the case where the slicing is performed before the demineralization, even when the resultant thin slice has a thickness of 1000 μm, the demineralization can be completed within 3 to 10 days by using any of the above-mentioned demineralizing solutions.

The EDTA solution that can be used as the demineralizing solution may be an aqueous solution of EDTA.2Na or EDTA.4Na. Although either an acidic EDTA solution or a neutral EDTA solution may be used, the acidic EDTA solution is preferably used when disinfection is performed simultaneously with the demineralization. It has been reported that the disinfection power of EDTA greatly varies with pH and this is because an acidic EDTA is more effective (pH 5.0 than pH 7.0) in disinfection against any bacterium (Kida, et al., Japanese Journal of Bacteriology, 47(4), 992). The demineralization with an EDTA solution can be performed using the aqueous solution having a concentration of 5% to 30%. The liquid temperature of the demineralizing solution can be 4° C. to 60° C. A period of time required for the demineralization differs depending on the concentration, liquid temperature and pH of the demineralizing solution and the size and shape of an extracted tooth, and therefore cannot be generalized, but, in the case where an extracted bovine tooth is demineralized by a neutral EDTA solution in a state in which the size or shape of the tooth is as it is, 11 weeks or more are usually required for the demineralization.

The demineralization using an inorganic acid achieves the highest demineralization speed, but carries the risk of partially denaturing protein, and accordingly there is a risk of decreasing the quality of collagen. However, in an autologous dentin transplant operation performed by the inventors, a DDM membrane obtained by demineralization using nitric acid has achieved good performance (more excellent in treatment outcome, compared with existing commercially available transplant materials and autologous transplant bones). There is a possibility that demineralization using an organic acid achieves a better quality of collagen, compared with demineralization using an inorganic acid. A neutral EDTA demineralizing solution is the mildest demineralizing solution, whereas demineralization using the neutral EDTA demineralizing solution takes much time. The neutral demineralizing solution is the best for maintaining the quality of collagen.

The degree of demineralization of a tooth can be checked using a device capable of evaluating X-ray permeability, such as a soft-X-ray photography device, manufactured by Softex, or an X-ray photography device. This is because mineral components are X-ray-impermeable.

The degree of demineralization is checked at any time, and at the time when an X-ray-impermeable portion disappears completely, it can be determined that demineralization has been completed. Alternatively, when mineral components (Ca and P) are not almost detected by electron probe micro-analysis (EPMA), it can be determined that demineralization has been completed.

In the method for producing a DDM membrane according to the present invention, the slicing may be performed prior to the demineralization or the demineralization may be performed prior to the slicing. In the case where the demineralization is performed before the slicing, the method for producing a DDM membrane according to the present invention includes the steps of: demineralizing an extracted bovine tooth to obtain completely demineralized dentin; and slicing the dentin thinly to obtain a DDM membrane. When the demineralization is performed before the slicing, thin pieces of the tooth can be produced without a slicing machine for hard materials.

In a preferable embodiment, the slicing can be performed before the demineralization. In this embodiment, the method for producing a DDM membrane according to the present invention includes the steps of: slicing an extracted bovine tooth thinly to obtain slices of the tooth; and demineralizing the slices of the tooth to remove all or almost all of minerals, thereby obtaining a DDM membrane. When the slicing is performed before the demineralization, a period of time required for the demineralization can be shortened. The demineralized extracted tooth is elastic and sometimes needs a technique for producing thin slices, but, it is not technically difficult to slice such demineralized extracted tooth thinly by using a slicing machine for hard materials.

An extracted bovine tooth that conforms to standards for biologically derived raw materials such as drugs is used, and in particular, an extracted bovine tooth for which necessary information to ensure quality and safety has been confirmed should be used. The extracted bovine tooth should be obtained from a supply source having neither bovine spongiform encephalopathy (BSE) nor other transmissible spongiform encephalopathy (TSE). Regarding extracted bovine teeth belonging to 12 to 15-month-old cattle, a deciduous molar has not been subjected to root absorption by an after coming permanent tooth, and therefore dentin of the deciduous molar can be utilized. Regarding extracted bovine teeth belonging to 20- to 30-month-old cattle, a deciduous tooth and a permanent tooth in the mixed dentition period can be used.

In another embodiment according to the present invention, an extracted bovine tooth is preferably a permanent tooth rather than a deciduous tooth in terms of size (both are similar in the quality of dentin and the tubule structure), and is particularly preferably a tooth of bovine at the age in months when a target tooth (a premolar or a molar) is matured (even a root apex portion thereof is completely formed).

An extracted bovine tooth can be stored by freezing (for example, at −4° C. to −20° C.) or at low temperatures (0° C. to 4° C.) after the extraction until the slicing and the demineralization. After the extraction, the extracted tooth can be sufficiently washed to remove blood and flesh, and stored by freezing (for example, at −4° C. to −20° C.).

The production method according to the present invention can further include the step of disinfection. By using an acid demineralizing solution for the demineralization, the disinfection can be performed simultaneously with the demineralization. Furthermore, the method can additionally include, for example, gamma-ray irradiation, dry-heating treatment performed as a treatment to remove and inactivate virus in a blood preparation, or low-pH liquid incubation.

The medical membrane material according to the present invention can be stored by freezing (for example, at a temperature of −20° C.), at low temperatures (for example, at a temperature of 4° C.), and/or by vacuum freeze drying. Even when the medical membrane material according to the present invention that has been subject to vacuum freeze drying is reconstituted using a liquid, the medical membrane material can have such sufficient mechanical characteristics for practical use and such sufficient flexibility that the medical membrane material has before freeze-dried. Furthermore, the medical membrane material according to the present invention can be sterilized by a non-heating sterilization technique used for sterilization of medical equipment, such as ethylene-oxide gas (EOG) sterilization or gamma-ray sterilization. The present invention can provide a kit including: a freeze-dried medical membrane material; and a liquid in an amount suitable for reconstitution. Examples of the liquid used for the reconstitution include, but not limited to, a physiological saline, sterile water, a phosphate buffer, and a solution containing a healing promoter such as FGF2.

[Potentiality of DDM as Medical Material]

Dentin of teeth is formed in such a manner that hydroxyapatite crystals of calcium phosphate deposit on a dentin matrix (a part that fills between dentinal tubules) containing collagen fiber as a principal component, and dentin is similar to bone in terms of components, but is a tissue different from bone. The biggest difference between dentin and bone is that bone is replaced (remodeled) by a new bone while continuously repeating resorption and formation, whereas dentin is never remodeled once formed. Bone is not merely a support tissue for supporting a body, but an important organ that regulates calcium metabolism in a living body. A decrease in the concentration of calcium in blood immediately causes elution of calcium from a bone, so that functions of the body are kept normal. In a bone, bone resorption by osteoclasts and osteogenesis by osteoblasts occur at any time (remodeling), and thus bones of the whole body are remodeled. Attempts have been made to demineralize and transplant bones for 50 years or longer (Ray, R D et al., J. Bone. Joint Surg., 39-A:1119-1128, 1957, Mitsumori, Transplant, 1:90-103.1966). However, the inventors found that teeth are superior as a biomaterial to bone.

Potentiality of teeth as a medical material will be studied through a comparison with bone.

In both a human tooth and a tooth of mammals other than humans, dentinal tubules run densely from the outer surface of dentin toward the center (pulp cavity). Dentinal tubules run very densely and all the tubules run in parallel without intersecting each other. With this structure, even when a tooth is processed into any shape (such as a membrane shape, a granular shape, or a block shape), dentin always become a dense transplant material having continuous pores. In contrast, in a cortical bone (compact bone), continuous pores are basically not present, and blood vessel cavities that allow blood vessels to pass therethrough are scattered. In some cases, the blood vessel cavities form a continuous structure, but the path is irregular and such structure appears at low frequency. Furthermore, bone cavities are also present in a bone, and these form a dead end structure and are not continuous pores. A transplant material made from bone eventually becomes a wall, and therefore is overwhelmingly disadvantageous for blood supply after a transplant (FIG. 2B).

Dentin collagen of teeth is more insoluble, compared with bone. In pepsin digestion in 0.01M-hydrochloric acid at 4° C., approximately 35% of collagen of an adult bovine bone was solubilized by digestion for 72 hours, whereas only 5.6% of collagen of dentin of an adult bovine tooth was solubilized. Regarding swelling properties, it was reported that insoluble collagen, such as skin and the Achilles tendon, swelled to 4 to 8 times its volume at pH 2, whereas insoluble collagen of an adult bovine bone swelled 1.2 times, and insoluble collagen of the adult bovine dentin did not swell at all (Y. Nagai and D. Fujimoto (ed.), Experimental Methods for Collagen, Koudansha Scientific, pp. 21-22).

### EXAMPLES

Based on the following examples, the present invention will be more specifically described, but not limited to these examples.

Material: mandibular molars (the first molar (M1) to the third molar (M3)) of cattle (Holstein, adult, female)

Material source: Experimental Farm, Field Science Center for Northern Biosphere, Hokkaido University (Example 3)

Material: mandibular (pre)molars (the second premolar (P3), the third premolar (P4), the first molar (M1)) of cattle (Holstein, 14-month-old, male)

Material source: Tokachi Food Center, JA Tokachi Shimizu (Examples other than Example 3)

### Example 1: Production of DDM Membrane

Production Method:

1) By using a micro-cutting machine BS-300CP (Meiwafosis Co., Ltd.), a bovine molar or premolar tooth was cut into plates having a thickness of 250 μm to 500 μm, in a direction in parallel to the major axis of the tooth as illustrated in the left figure of FIG. 2A.

2) The plate-shaped cut tooth was immersed in a nitric acid demineralizing solution (inorganic acid) (2 v/v % nitric acid, pH 0.5) to be demineralized. By using a soft-X-ray photography device manufactured by Softex, the degree of the demineralization was checked at any time, and at the time when an X-ray-impermeable portion disappears completely, it was determined that the demineralization was completed. It took three days to complete the demineralization of the plate-shaped molar having a thickness of 500 μm. When the plate produced from the molar and having a uniform thickness was completely demineralized, a flexible membrane-shaped structure with rubber-like elasticity was created. This was taken as a DDM membrane. The thus-produced DDM membrane was stored in a 0.1M tris-hydrochloric acid solution (pH 7.5) until used.

### Example 2: Freeze-dried DDM Membrane

The DDM membrane (500 μm in thickness) produced in Example 1 was freeze-dried using a vacuum freeze dryer (VD-400F freeze dryer, TAITEC CORPORATION) in accordance with a manual.

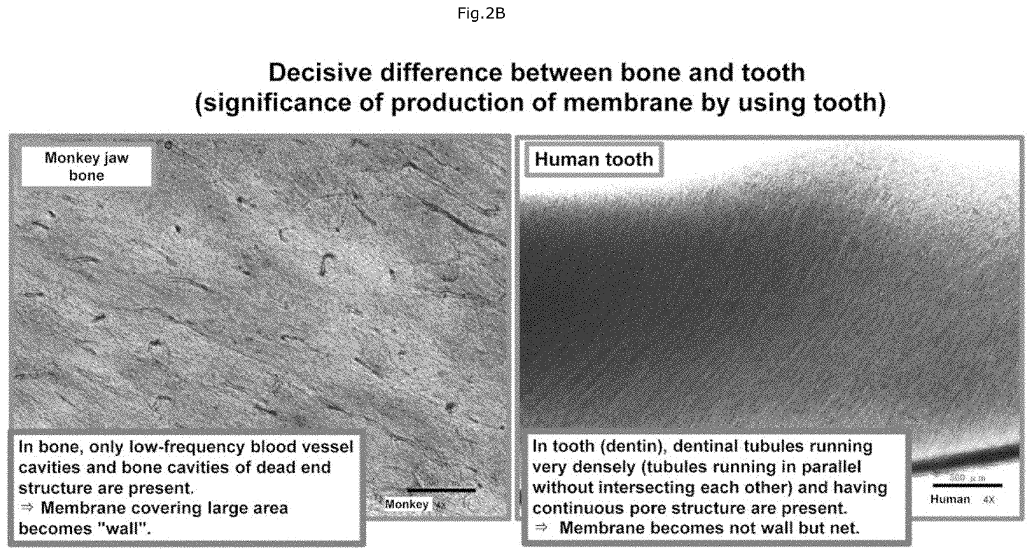

The freeze-dried DDM membrane was immersed in PBS(−) to be reconstituted as illustrated in FIG. 3. According to palpation, the reconstituted freeze-dried DDM membrane had no change in strength and flexibility when stretched, compared with the DDM membrane before freeze-dried.

### Example 3: Comparison Between Demineralization with Neutral Demineralizing Solution and Demineralization with Weak Acid Demineralizing Solution

Bovine mandibular molars (M1 to M3) and bovine anterior teeth (as for EDTA, including one piece of human molar by reference) were immersed in a neutral EDTA demineralizing solution (10 w/v % EDTA.2Na aqueous solution, pH 7.4) or a weak acidic formic acid demineralizing solution (5 v/v % formic acid aqueous solution, pH 5.0), as they were. By using a soft-X-ray photography device manufactured by Softex, soft-X-ray photographs were taken every week.

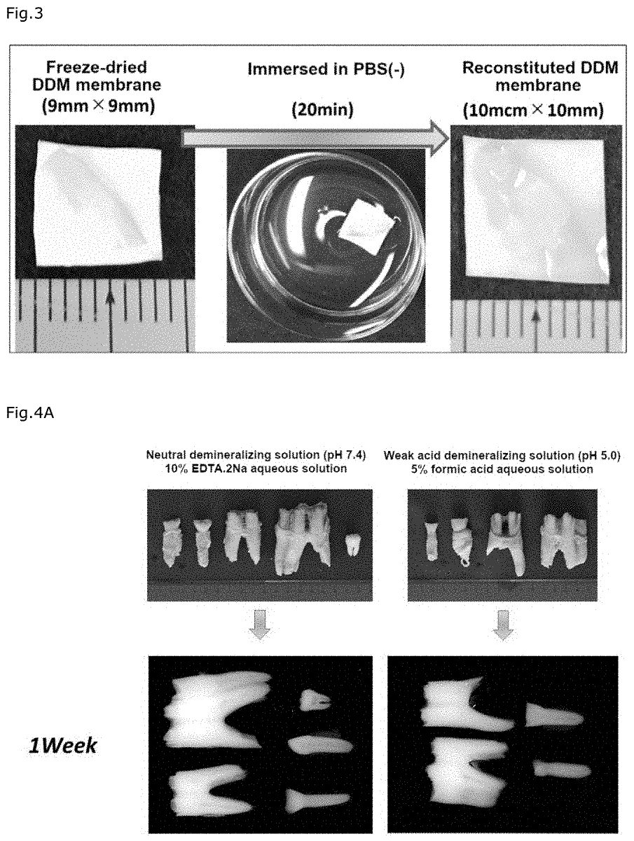

The soft-X-ray photographs are illustrated in FIG. 4. Until complete demineralization in which an X-ray-impermeable portion has disappeared, the demineralization using the weak acid demineralizing solution took 6 to 14 weeks, whereas the demineralization using the neutral demineralizing solution took 11 to 24 weeks.

### Example 4: Comparison Between Tooth and Bone

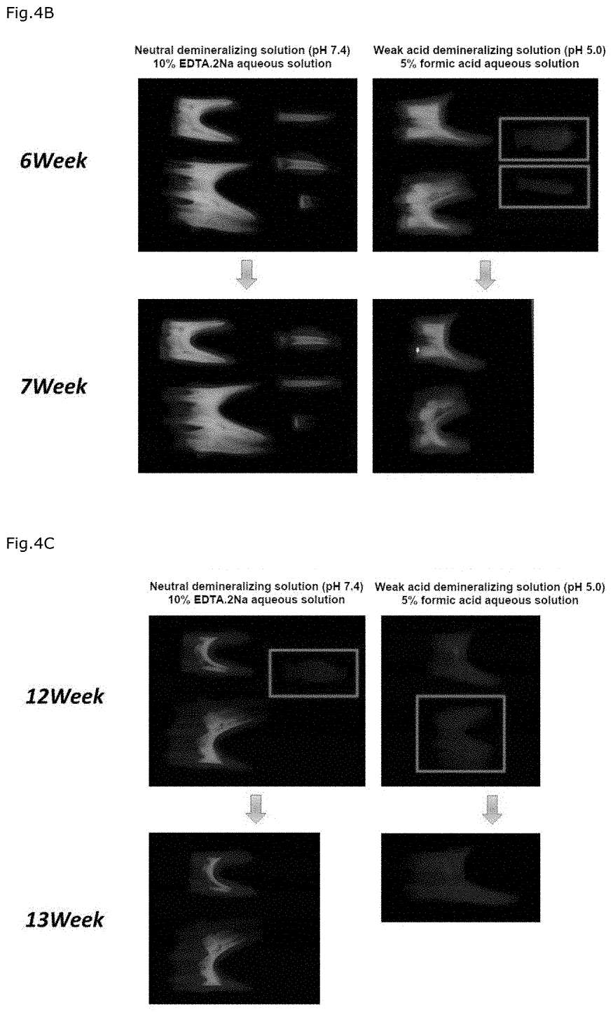

As a material of the membrane, a tooth (bovine molar) and a bone (bovine alveolar bone) were compared. The tooth and the bone were demineralized by the same method, and thin-sliced membranes thereof were prepared. Specifically, by using a weak acidic formic acid demineralizing solution (5% formic acid aqueous solution, pH 5.0), a bovine mandible was demineralized, and, by using a microtome for preparing tissue slices: RETORATOME REM-710 (Yamato Kohki Industrial CO., LTD.), the demineralized molar and the surrounding demineralized alveolar bone were thinly sliced to produce thinly sliced membranes. Photographs of the produced slices are illustrated in FIG. 5. The demineralized bone was dried and crumbly, and accordingly it was difficult to produce the slices. If a slice was forcibly produced, the slice needed to have a thickness of approximately 2 mm.

When the slices derived from the bone were bent, the slices were broken and had no flexibility. In contrast, the slices produced by demineralizing the tooth could have even a thickness of 10 μm. Even when the slices were produced so as to be ultrathin, the slices were thin slice membranes being very flexible and hardly broken and capable of being sutured.

### Example 5: Usage Example 1 of DDM Membrane as Base Material for Cell Proliferation

1) Preparation of DDM Membrane

A bovine mandibular anterior tooth was horizontally cut to a thickness of 250 μm by using a micro-cutting machine BS-300CP, and then completely demineralized by a weak acidic formic acid demineralizing solution (5% formic acid aqueous solution, pH 5.0) to prepare a DDM membrane. The reason why the DDM membrane obtained by horizontally cutting the bovine mandibular anterior tooth was used is that the DDM membrane is similar in shape and area to the bottom of wells of a 24 well-plate base on which a cell proliferation test was conducted.

2) Preparation of Periodontal Ligament Stem Cell

There was used a human periodontal ligament mesenchymal stem cell extracted by a method described in an international application publication (WO2019/074046 A1, the contents of which is incorporated herein by reference in its entity) that claims priority to Japanese Patent Application No. 2017-198072).

3) Cell Proliferation Test

The DDM membrane is composed of Type I collagen, and therefore theoretically can be expected to have a promotion effect on cell proliferation via Type I collagen (for example, by integrin signaling).

In order to confirm whether the present DDM membrane has the above-mentioned function, a group of the DDM membranes coated with commercially available collagen (collagen-coated group) and a group of the DDM membranes not coated with collagen (untreated group) are set, and, for the purpose of confirming whether the group of only the DDM membranes achieves the same cell proliferation effect as that of the commercially available collagen-coated group the function of which has been already assured, a proliferation test of periodontal ligament stem cells was conducted. As the collagen-coated group, there was used a group of the DDM membranes coated with Type I collagen by using Cellmatrix (registered trademark) Type I-C(Nitta Gelatin Inc.) in accordance with a protocol described in its manual.

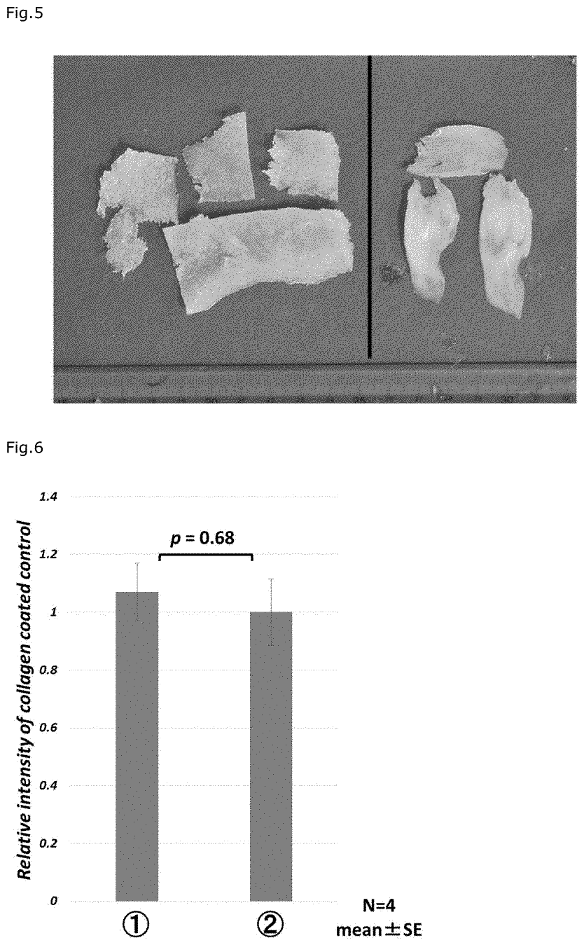

On wells of a 24 well plate, the DDM membranes and the collagen-coated DDM membranes were placed. The periodontal ligament stem cells were seeded at 1.3×10⁴ cells/well, and incubated in 10 v/v %-FBS-containing DMEM/F-12 (Sigma) as a basal medium for three days under the conditions of 5% CO₂ and 5% O₂ at 37° C. After the incubation, a cell proliferation test was conducted using Cell Counting Kit-8 (DOJINDO LABORATORIES) in accordance with a protocol described in its manual. FIG. 6 illustrates the results. It was confirmed that the DDM membranes had cell proliferation potency equal to or higher than that of the collagen-coating DDM membranes.

This experiment indicates a possibility that the DDM membrane could maintain a cell proliferation promotion effect that Type I collagen has.

### Example 6: Usage Example 2 of DDM Membrane as Base Material for Cell Proliferation

1) Immersion of DDM Membrane in FGF2

The DDM membrane is composed of Type I collagen. Type I collagen has the characteristic of adsorbing protein including a matrix binding protein group (for example, FGF and BMP). Furthermore, because of the presence of an infinite number of dentinal tubules, the surface area of this collagen matrix is considerably increased, and the DDM membrane adsorbs the protein also inside the tubules, thereby adsorbing and holding a large amount of cytokine even with a thin membrane structure, so that the effect of sustained-release of cytokine after transplant in an affected site can be expected.

In order to confirm the above-mentioned function, the DDM membrane prepared at 1) of Example 5 was immersed in a basal medium (10% FBS-containing DMEM/F-12 (Sigma)), and incubated for one day, which was taken as a control group. On the other hand, the DDM membranes were immersed in the basal media containing a growth factor FGF2 (RandD) at concentrations of 50 ng/mL and 200 ng/mL, respectively, and incubated for one day, which were taken as experimental groups. The DDM membranes of the control group and the two experimental groups were washed well with PBS(−) (Sigma) 5 times, and not-adhering FGF2 was sufficiently washed away, and then the DDM membranes were placed on the bottom of a 24 well-plate.

2) Cell Proliferation Test

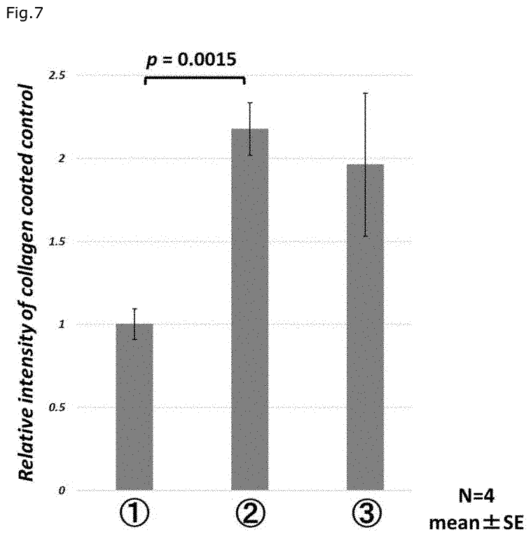

The periodontal ligament stem cells were seeded at 1.3×10⁴ cells/well, and incubated in a basal medium (10% FBS-containing DMEM/F-12 (Sigma)) for three days under the conditions of 5% CO₂ and 5% O₂ at 37° C. After the incubation, a cell proliferation test was conducted using Cell Counting Kit-8 (Dojindo Laboratories) in accordance with a protocol described in its manual. FIG. 7 illustrates the results. It was confirmed that the experimental groups (FGF2-impregnation groups) exhibited more remarkable cell proliferation activity having a significant difference, compared with the control group (untreated group).

This experiment revealed that, when the DDM membrane was immersed in advance in a cytokine solution, any cytokine was allowed to adhere onto the DDM membrane, and there was a possibility that not only the addition of the function shown in Example 5 but also the addition of the function shown in Example 6 could contribute to the great enhancement of regenerative ability.

### Example 7: Usage Example 3 of DDM Membrane as Base Material for Cell Proliferation

The DDM membrane (250 μm in thickness) completely demineralized by a demineralizing solution (aqueous solution) containing 10% nitric acid, 10% formic acid, or 10% EDTA (neutral) was placed on the bottom of a culture dish, and a predetermined number of periodontal ligament stem cells (1.3×10⁴ cells) were seeded and cultured under the conditions of 5% CO₂ and 5% O₂ at 37° C. On the DDM membrane demineralized by EDTA, good cell-adhesion was observed on the second day of the culture, and even when the DDM membrane was shaken strongly, the cells were not detached from the DDM membrane. On the DDM membrane demineralized by nitric acid and the DDM membrane demineralized by formic acid, good cell-adhesion was observed on the fifth day of the culture, and even when the DDM membranes were shaken strongly, the cells were not detached from the DDM membranes.

Thus, the DDM membrane demineralized by EDTA most strongly maintained the characteristics of collagen. There is a report to support this that, a matrix obtained by demineralization using nitric acid does not show a typical collagen pattern by X-ray analysis as is shown in a matrix obtained by demineralization using EDTA (Urist M R A K, et al., Clin Orthop Relat Res. 1965 May-June; 40:48-56), and the experiment results supported the above-mentioned report. On the other hand, there is a report that, in the case of xenogeneic transplant, an EDTA demineralization method made antigenicity maintained, whereby inflammation was induced in a transplant site (Mitsumori, Transplant, 1:90-103.1966). From the viewpoint of maintaining the characteristics of collagen, a possibility that EDTA demineralization was the optimal method for treating a transplant material was indicated. In contrast, it is said that the disinfection activity and the endotoxin deactivation activity of EDTA are considerably weaker, compared with the disinfection activity of inorganic acid or organic acid. From the viewpoints of disinfection activity and xenogeneic transplant, there is a possibility that an inorganic acid is preferably used for a demineralizing solution for a transplant material to be transplanted into a living body.

### Example 8: Usage Example 1 of DDM Membrane as Transplant Material

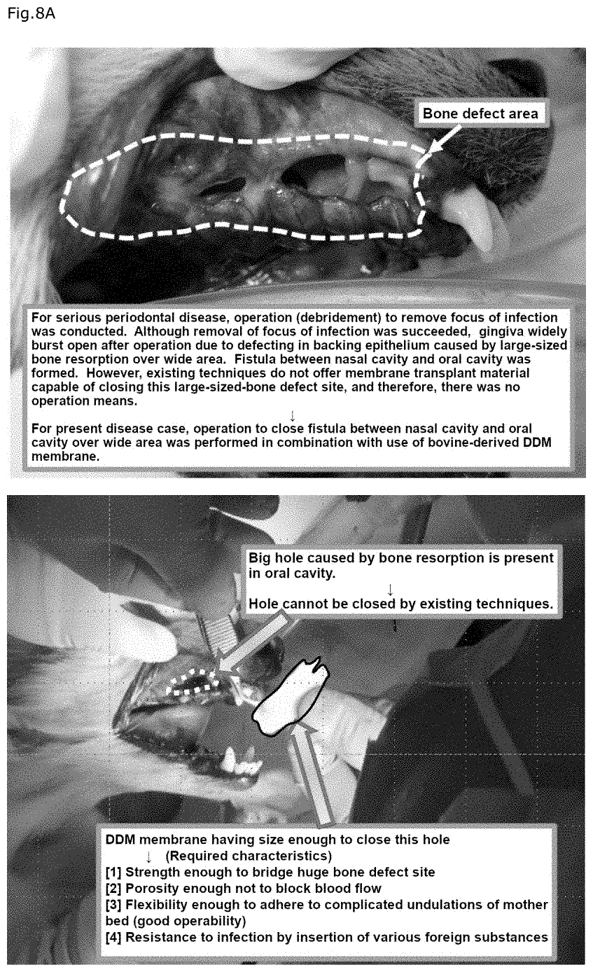

By using a DDM membrane, an operation was conducted to close an affected site of a dog in which marked bone resorption had occurred in a maxilla bone in a wide area due to a serious periodontal disease. FIG. 8 illustrates photographs taken at the time of the operation of the present example.

Preparation of DDM membrane: Before use, the DDM membrane (500 μm in thickness) produced in Example 1 was neutralized using a 0.1M tris-hydrochloric acid solution (pH 7.5), and then used for transplant. Fiblast Spray 250 (containing 250 μg of trafermin) as a FGF2 preparation was adjusted to 100 ng/mL, and the neutralized DDM membrane was immersed therein, and incubated overnight at 4° C. After washed with a physiological saline solution, the DDM membrane was used for transplant.

Information on affected animal: 17-year-old female miniature dachshund suffering from a serious periodontal disease throughout its maxilla (jaw size: small because of a small dog, having a pointed muzzle). After a risk and the likes were sufficiently explained to an owner and informed consent was obtained from the owner, a DDM membrane transplant operation was conducted.

Situation of previous operation: The general status was worsened by pus discharge from molar pockets and severe inflammation due to a serious periodontal disease. For this disease case, extraction of all teeth of maxillary molar sites on both sides and curettage (debridement) of a periodontal disease inflammatory wound were performed under general anesthesia. Although bone resorption had been progressed faster than expected, the curettage succeeded in removing the focus of inflammation as much as possible, causing the affected animal to have no bone to separate an oral cavity from a nasal cavity in a wide area, i.e., to have a large hole in the oral cavity. After that, a relaxing incision was made to draw up a gingiva on the cheek side, and a wound was closed using an absorbable surgical suture (VICRYL RAPID (registered trademark): Ethicon).

The inflammatory condition was dramatically improved and the affected animal recovers its energy. However, bone backing was not present in a wide area on both sides due to the serious bone resorption, and therefore, a gingiva in the closed site burst open two weeks after the operation, so that the oral cavity and the nasal cavity widely communicated with each other (see the upper figure of FIG. 8A). Properly, another operation to block the communication between the oral cavity and the nasal cavity (an operation to close a fistula between the nasal cavity and the oral cavity) should have been conducted, but, the existing therapies did not have any effective closing method for a status in which there was no backing epithelium due to a bone defect extending over a wide area.

Transplant operation of DDM membrane: Without a choice, a transplant operation was performed after informed consent was obtained from the owner, in which a DDM membrane produced from cattle was placed, as a backing, inside gingivae and fixed by suturing the DDM membrane into the gingivae at the time of suturing the gingivae together. The used DDM membrane was 20 mm×40 mm in size. This size was enough to cover a site in which the oral cavity and the nasal cavity completely communicated with each other (see the lower figure of FIG. 8A and the upper figure of FIG. 8B).

Furthermore, the DDM membrane has the original characteristics, which were not achieved by conventional transplant materials, such as (1) having mechanical strength enough to bridge a bone defect site extending over a wide area, (2) making it possible to avoid a blood flow disorder by the porosity (see FIG. 1) owing to the dentinal tubule structure, although, in the case of a conventional membrane transplant material, a possibility is strongly suggested that covering with the membrane transplant material in a wide area like the present disease case causes a blood flow disorder over a wide area and the covered mucous membrane becomes necrotic at an early stage, and (3) achieving a regeneration promotion effect by advance impregnation with the FGF2 preparation capable of promoting healing of an affected site, thereby, unlike the previous case, any gingiva did not burst open after the operation, the inflammation entirely subsided, gingivae other than a gingiva around a remaining tooth conglutinated, and epithelization was completed (see the lower figure of FIG. 8B).

With this disease case, there was found out a possibility of a new therapy for a difficult disease case that could not be healed with the existing therapy. Furthermore, it was confirmed that, in the results of a blood test conducted on the 21st day after the operation, the number of leucocyte decreased from 33570/μL, measured before the operation, to 15550/μL, and the value of CRP (C reaction protein) serving as an inflammation marker decreased from 7 mg/dL, measured before the operation, to 0.6 mg/dL, and thus rejection symptoms due to xenogeneic transplant did not appear.

### Example 9: Usage Example 2 of DDM Membrane as Transplant Material

Confirmation of Overcoming Low Resistance of the Existing Transplant Material to Infection which is Pointed Out in the Existing Technologies

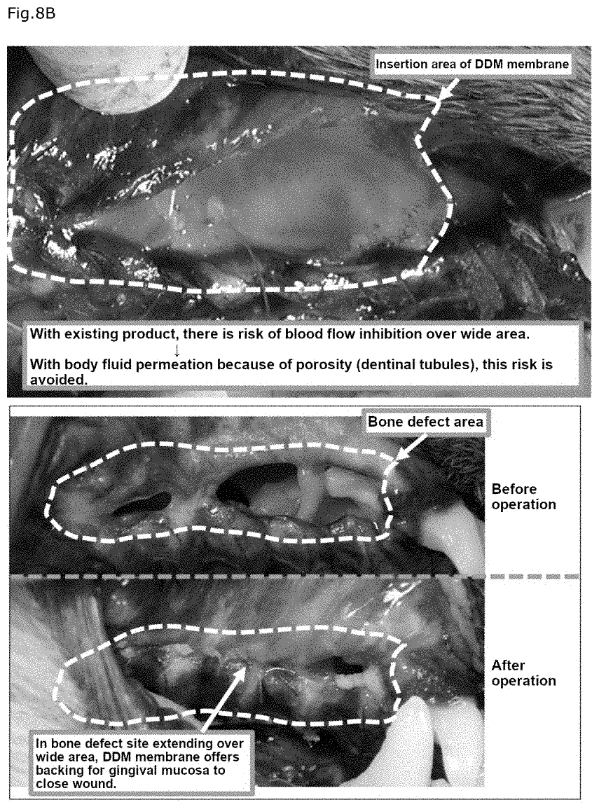

In an animal experiment using miniature swine, the DDM membrane was used as a membrane for prevention of infection. In the present example, the thickness of the DDM membrane was set at 2000 μm in order to maintain the DDM membrane for a long term. It was studied whether the DDM membrane functioned as a membrane for prevention of infection under unclean conditions, by transplanting the DDM membrane not into the interior of a wound site (inside tissue), but into the exterior of the wound site. FIG. 9 illustrates photographs taken at the time of an operation in the present example.

The DDM membrane (approximately 5 cm² in size: 4 cm×1.3 cm) was produced by completely demineralizing bovine molars with an inorganic acid. Operation targets were four mandibular anterior teeth. After an incision was made in gingival sulci, gingivae were exfoliated in all layers to form gingival flaps, so that a bone was exposed. The teeth were extracted without breaking an alveolar bone. Thus, extraction sockets (bone defect) were formed. This bone defect (hole) was filled with a filling material (bovine-derived DDM granules), and covered with the above-described DDM membrane, and the gingival flaps were returned on the DDM membrane and sutured with an absorbable surgical suture, whereby the DDM membrane was fixed just under the gingival flaps, and bone regeneration was evaluated. The DDM membrane was fixed by suture (see the upper figure of FIG. 9).

The miniature swine did not have the intention of taking good care of an operative site (an open wound) and put powder food into its oral cavity in the manner of collecting the powder food with gingivae of a mandibular anterior tooth site (the operative site) to eat the powder food, so that food residues adhered to the operative site (the open wound), and hence, a high risk of infection of deep bone tissue was predicted. As predicted, 3 days after the transplant, food residues were allowed to adhere around the DDM membrane exposed as illustrated in a photograph (the center figure of FIG. 9), but, any sign of inflammation was not observed in the surrounding gingivae.

9 days after the transplant, the DDM membrane with dirt due to the food residues fell off together with the suture. Just under the place at which the DDM membrane fell off, epithelization was completed, and thus, covering with the DDM membrane completely prevented infection during an unstable stage under unclean environments (see the lower figure of FIG. 9). It has been pointed out that a conventional graft is weak against infection, whereas it was sufficiently suggested that the DDM membrane according to the present invention was highly resistant to infection.

There were performed 4 types of experiments: in addition to the above-described experiment including: filling with the DDM granules; and covering with the DDM membrane, 1) an experiment not including filling with the DDM granules, but including covering with the DDM membrane, 2) an experiment including: filling with the DDM granules; and covering with the DDM membrane impregnated with FGF2, and 3) an experiment including: filling with the DDM granules; and covering with the DDM membrane reconstituted with PBS(−) after freeze-dried. Any of the experiments showed no findings indicating a bacterial infection.

A CT inspection and a histological study on bone regeneration were performed 3 weeks after the transplant, and as a result, in the experiments 2), 3), and 4), new bone formation to a height almost enough to fill an extraction socket was observed. In the experiment 1), the level of new bone formation was lower compared with other cases, but a juvenile new bone began to be induced. In all the disease cases, histological analyses showed no sign of postoperative bacterial infection, such as inflammatory cell infiltration.

### Example 10: Usage Example 3 of DDM Membrane as Transplant Material

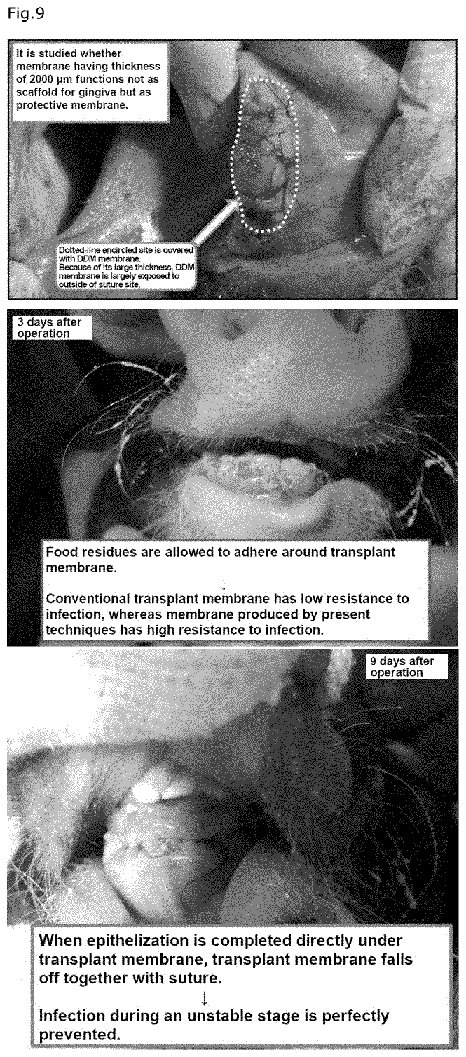

The DDM membrane was applied to a dog suffering a serious mandibular fracture. The affected animal was 15-year-old female miniature dachshund, and, in its mandibular alveolar bone, a decrease in bone density that was associated with serious bone resorption due to a serious periodontal disease was observed. When a periodontal therapy was given after informed consent was obtained from an owner, a serious mandibular fracture to the extent of reaching a mandibular inferior border occurred at two portions around molars (indicated by arrows in FIG. 10a and FIG. 10b). Considering that there was heavy bleeding and the affected animal was of an advanced age, after owner's consent was obtained, a treatment was applied in which the DDM membrane was transplanted into the periosteum (into a gingiva) and covering with the DDM membrane (approximately 5 cm² in size and 500 μm in thickness) so as to cover a fracture line (see FIG. 10c).

The DDM membrane came into intimate contact with the fracture portions immediately after the covering, so that the amount of bleeding decreased. After that, an anterior tooth serving as a support tooth was temporarily fixed (fixed for a while) to an affected tooth, whereby a mesial part and a distal part of the fractured jaw were fixed to each other (intermaxillary fixation), and the wound was closed. 1.5 months after the operation, extinction of the fracture line was observed (see FIG. 10d). In consideration of situations, such as the affected animal's age in month and bone density, it was considered that the DDM membrane acted effectively for bone regeneration in the fracture portions.

### Example 11: Usage Example 4 of DDM Membrane as Transplant Material

In surgical operations on digestive organs, stapled digestive tract anastomosis using an automatic suture apparatus in place of hand-suturing has been widely used. Digestive tract anastomosis by hand-suturing is performed by joining submucosal layers in which abundant blood vessels are present. By contrast, stapled digestive tract anastomosis is performed by connecting mucosal layers in which few blood vessels are present, leading to the problem that some of the mucosae do not conglutinate well, and a leak (the leak of the contents) occurs with a probability of approximately 10% (Bertelsen C A, et al., Colorectal Dis. 2010 July; 12: e76-81).

In order to confirm that the DDM membrane can be applied also to the digestive tract surgery field, a test was conducted in which the DDM membrane was applied to a digestive tract anastomotic site of swine. The test was conducted in Toya laboratory of Hokudo Co., Ltd. As an affected animal, a livestock swine (LWD type, four-month-old, female, 45 kg in weight) having a digestive tract similar in size to a human digestive tract was used. Laparotomy was performed for the affected animal under anesthesia, and anastomoses of the large intestine and the small intestine were performed, as follows.

1) Large Intestinal Anastomosis

An anastomosis of the large intestine was performed by using an automatic anastomosis apparatus PROXIMATE (registered trademark) ILS CDH25 (Ethicon). This apparatus has a cylindrical knife and metal staples in a staple housing having a trocar. Purse-string suture is put in an end of each of digestive tracts to be anastomosed, and then the apparatus body is inserted into one intestinal tract to make the trocar exposed and an anvil was inserted into the other intestinal tract, and the trocar was coupled to the anvil. After that, the staples are fired from the staple housing by operating a firing handle to form a circular staple line, and at the same time, a tissue inside the staple line is excised in a ring shape by the cylindrical knife, whereby the anastomosis of the intestinal tracts is performed (PROXIMATE (registered trademark) ILS package insert (Japanese medical device approval number: 21900BZX00879000); Ethicon, Inc., Endoscopic Curved Intraluminal Stapler, Instructions For Use).

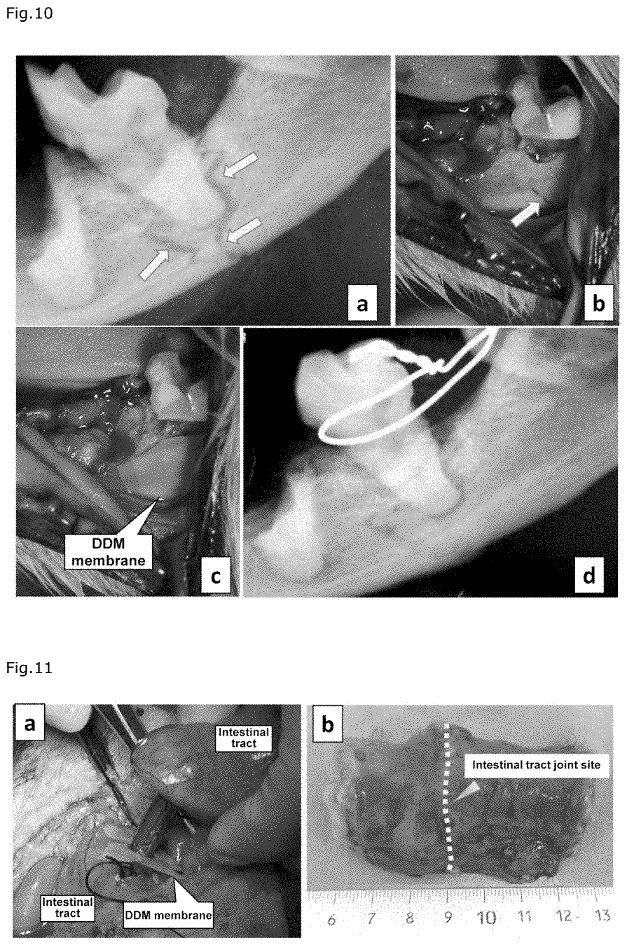

After ends of the large intestine were coupled to each other by the trocar and the anvil, two sheets of the DDM membrane (approximately 5 cm² in size, 500 μm in thickness) were interposed therebetween (FIG. 11a), and a firing was conducted, so that end-to-end anastomosis of the oral cavity side and the anus side of the large intestine was performed via the DDM membrane, and then the abdomen of the swine was closed. One week after the operation, a large intestinal anastomosis site was extracted and subjected to an anastomotic bursting pressure (ABP) test. The anastomotic bursting pressure test is a method performed in such a manner that an intestine is excised approximately 5 cm front and back from the center of an anastomosis site, and one end of the excised intestine is sutured as it is with a suture, whereas the other end thereof is fixed with a suture to a hose connected with a pressure gauge, and after that, air is sent in from the hose to inflate the intestine underwater. A pressure at the time when a leak occurs from the anastomosis site and an air bubble is observed underwater is recorded, and this pressure is regarded as a measured value.

FIG. 11b illustrates a photograph of the rectal anastomosis site. In the anastomosis site, good adhesion was observed in appearance. When an anastomotic bursting pressure test was conducted, a normal intestinal tract site burst earlier under a pressure load of approximately 300 mmHg than the anastomosis site did, and therefore, a pressure test at 300 mmHg or higher could not be conducted for the anastomosis site. According to a previous paper in which the anastomosis of the swine large intestine was performed using the same automatic anastomosis apparatus (Vanbrugghe C, et al., Surg Innov. 2017 June; 24(3):233-239), an intestinal tract leak occurred at 50 to 180 mmHg, and in consideration of this, it was indicated that the DDM membrane prevents an intestinal tract leak by promoting adhesion and making firm-joining in the large intestinal anastomosis site.

2) Small Intestinal Anastomosis

By using an automatic suture apparatus, namely, DST Series (registered trademark) GIA (registered trademark) stapler, anastomosis of the small intestine was performed. This apparatus has a staple cartridge having a knife within and an anvil. An intestinal tract is sandwiched between the staple cartridge and the anvil, and then staples are fired from the staple cartridge by operating a firing knob to form two groups of linear staple lines, and at the same time, cutting is made between one group of the linear staple lines and the other group by the knife, and the anastomosis and cutting of the intestinal tract is thus performed.

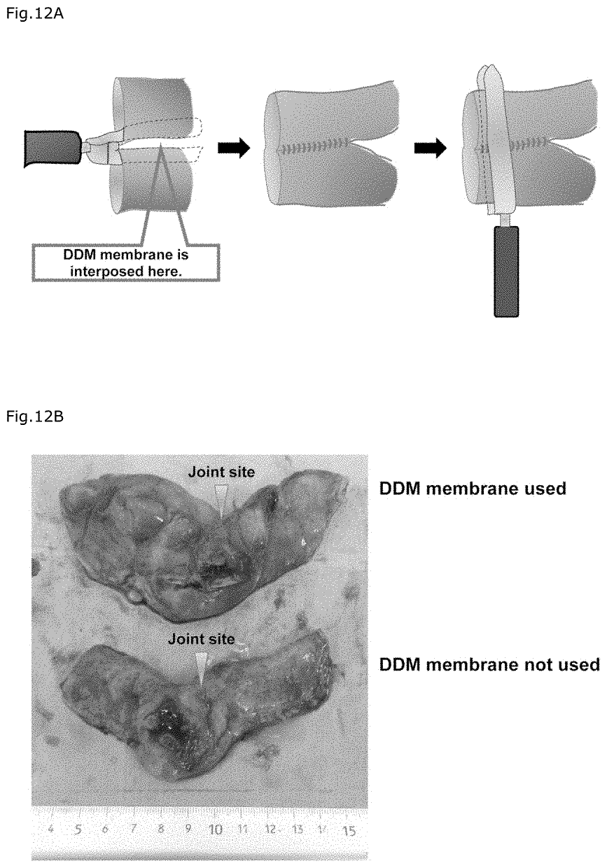

Two anastomosis target sites were set in the small intestine, and, as illustrated in FIG. 12A, side-to-side anastomosis to anastomose the side faces of the intestine was performed. First, the small intestine was cut around the anastomosis target sites, then the cut faces were lined up side by side, and suturing and cutting were performed with or without the DDM membrane (approximately 5 cm² in size and 500 μm) interposed. Subsequently, cut ends were sutured to complete the anastomosis, and then, the abdomen of the swine was closed. One week after the operation, two small intestinal anastomosis sites were extracted and subjected to an anastomotic bursting pressure test.

FIG. 12B illustrates a photograph of the small intestinal anastomosis sites. In both the anastomosis sites in one of which the DDM membrane was used and in the other of which the DDM membrane was not used, good adhesion was observed in appearance. When an anastomotic bursting pressure test was conducted, the anastomosis site in which the DDM membrane was not used burst at 95 mmHg, whereas, in the anastomosis site in which the DDM membrane was used, a minute leak that caused the generation of minute bubbles was observed at 170 mmHg. It is said that an intestinal tract anastomosis site anastomosed with an automatic suture apparatus often causes a leak within one week after operation, and sometimes bursts due to flatus. It was confirmed that the small intestinal anastomosis site in which the DDM membrane was interposed was highly resistant to a pressure of higher than approximately 100 mmHg, the pressure being estimated to be applied to an intestinal tract at the time of flatus, and hence, it was indicated that the DDM membrane prevented an intestinal tract leak by promoting adhesion and making firm-joining in the small intestinal anastomosis site.

### INDUSTRIAL APPLICABILITY

The present invention is useful in the medical field.

1-9. (canceled)

10. A method for producing a medical membrane material, the method comprising:
slicing and demineralizing an extracted bovine tooth to obtain a demineralized dentin matrix (DDM) having an area in a range of 2 cm² to 50 cm², having a plate or membrane shape, and being completely demineralized, wherein
the slicing is performed prior to the demineralization or the demineralization is performed prior to the slicing.

11. The method according to claim 10, wherein the demineralization is performed by immersing the extracted tooth in a demineralizing solution that is an aqueous solution of any of an inorganic acid, an organic acid, and ethylenediaminetetraacetic acid (EDTA).

12. A method for treating human or a non-human animal having an affected site, the method comprising:
bringing a medical membrane material into intimate contact with the affected site,
thereby protecting, reinforcing, or bonding the affected site,
wherein the medical membrane material is a demineralized dentin matrix (DDM) derived from an extracted bovine tooth, the medical membrane material has an area in a range of 2 cm² to 50 cm², has a plate or membrane shape, and is completely demineralized.

13. The method according to claim 12, wherein the medical membrane material has a continuous surface.

14. The method according to claim 12, wherein a drug is applied to the medical membrane material or the medical membrane material is impregnated with the drug.

15. The method according to claim 12, wherein the affected site is a wound site or an injury site.

16. The method according to claim 12, further comprising, prior to bringing the medical membrane material into intimate contact with the affected site:
covering the affected site with the medical membrane material.

17. The method according to claim 12, further comprising, prior to bringing the medical membrane material into intimate contact with the affected site:
filling the affected site with a filler, a drug, or a mixture of the filler and the drug; and
covering at least a part of the filler, the drug, or the mixture of the filler and the drug filled in the affected site, with the medical membrane material.

18. The method according to claim 12, further comprising, prior to bringing the medical membrane material into intimate contact with the affected site:
connecting the affected site via the medical membrane material.
